(19) United States
(12) Patent Application Publication
(10) Pub. No.: US 2005/0244347 A1
(43) Pub. Date: Nov. 3, 2005

(54) Oral care products comprising calcium phosphates

(76) Inventors: Dev K. Mehra, Furlong, PA (US); Michael C. Withiam, Landenberg, PA (US); John M. Cornelius, Forest Hill, MD (US)

Correspondence Address: Carlos Nieves, Esq.;J. M. Huber Corporation, 333 Thornall Street, Edison, NJ, 08837-2220, US

(21) Appl. No.: 10/837,384

(22) Filed: Apr. 30, 2004

(51) Int. Cl. A61K 7/16; A61K 9/20

(52) U.S. Cl. 424/57

(57) ABSTRACT

A rapidly disintegrating oral care tablet is provided. The tablet comprises: a calcium phosphate; a super disintegrant; and a sugar alcohol. When immersed in water the tablet has a friability of less than about 2% and disintegrates in less than about 60 seconds.

### BACKGROUND OF THE INVENTION

Many consumer products, such as health and personal care products, are manufactured and packaged in solid, compacted form. The solid, compacted product form has several advantages over other product forms, such as relative ease of manufacture and durability in packaging and shipment and convenience in use and in storing for retailers and consumers alike. The compressed tablet form is particularly well-suited for oral care and hygiene.

However, in certain situations it would be beneficial if the tablet would disintegrate in the mouth so that tooth cleaning could be affected without the necessity of having access to a toothbrush or to water. For example, hikers, campers, boaters, or people traveling or eating in public places, could use an oral care tablet that rapidly disintegrates in the mouth providing a convenient and effective solid form delivery system for tooth cleaning and mouth freshening.

Unfortunately, most tablets do not readily disintegrate in the mouth, but instead disintegrate in a slow and uneven fashion, for example when chewed. Given the forgoing there is a continuing need for solid form oral care preparations that rapidly disintegrate in the mouth and that are not friable under packaging and shipping conditions.

### BRIEF SUMMARY OF THE INVENTION

The present invention includes a rapidly disintegrating oral care tablet comprising (a) about 10% to about 80% calcium phosphate, (b) about 20% to about 80% of a sugar alcohol and (c) about 1% to about 30% of a super disintegrant.

### DETAILED DESCRIPTION OF THE INVENTION

All parts, percentages and ratios used herein are expressed by weight unless otherwise specified.

All publications, patent applications and issued patents mentioned herein are hereby incorporated in their entirety by reference.

The present invention relates to personal care products that are oral care products in solid or semi-solid form such as dentifrices, toothpastes, and breath-fresheners; these personal care products may include calcium phosphates.

The oral care products of the present invention typically contain from about 10% to about 80% calcium phosphate, preferably from about 15% to about 50%, about 20% to 80% sugar alcohol, preferably about 20% to about 70%, and about 1% to about 30% of a super disintegrant, preferably about 3% to about 15%, more preferably about 3% to 5%.

Calcium phosphate provides dual functionality to the rapidly disintegrating oral care tablets. Calcium phosphate is a water insoluble substance, which in the presence of a super disintegrant enables very rapid tablet disintegration when the tablet contacts water. Additionally, calcium phosphate serves as a dental abrasive providing tooth cleaning and polishing. Suitable calcium phosphates of the present invention include dicalcium phosphate, also known as dibasic calcium phosphate, both anhydrous (DCP) and dihydrate (DCPD) forms; tricalcium phosphate (TCP), also known as tribasic calcium phosphate; calcium pyrophosphate; calcium polyphosphate and the like, and combinations of more than one calcium phosphate.

The sugar alcohol provides multiple functions to the rapidly disintegrating oral care tablet. The sugar alcohol provides good aesthetic properties to the dissolved oral care tablet such as taste (sweetness and coolness due to its endothermal heat of solution) and “mouth texture” or body; aids in rapid tablet disintegration; and serves as a tablet filler. Suitable sugar alcohols are those given in The Encyclopedia of Chemical Technology, Vol. 23, 4^{th} Edition, Mary Howe-Grant, editor, John Wiley & Sons, New York, N.Y. (1997) pages 93-113, which is incorporated herein by reference, and include erythritol, xylitol, sorbitol, maltitol, mannitol, lactitol, and the like, used singly and in combinations, with mannitol and sorbitol preferred.

The super disintegrant facilitates the break-up of a tablet when it is placed in an aqueous environment, such as the mouth. Super disintegrants in contact with water swell, wick-in water or otherwise provide a disruptive force to a tablet causing it to break apart. Suitable super disintegrants include one or more of sodium starch glycolate, available as e.g. Explotab and Explosol; croscarmellose sodium (cross-linked sodium carboxymethyl cellulose) available as e.g. Ac-Di-Sol® and Nymcel® ZSX; and cross-linked polyvinylpyrolidone available as e.g., Polyplasdone XL.

In addition to the aforementioned ingredients, the oral care products of the present invention may also include several other ingredients such as additional disintegration aids, organoleptic enhancers, additional abrasives, thickening agents, (also sometimes known as thickeners, binders, gums, or stabilizing agents), therapeutic agents, and preservatives.

These solid formed oral care preparations may also include one or more disintegration aids, in addition to the super disintegrant. Suitable disintegration aids include natural, modified or pregelatinized starch; natural or chemically-modified cellulose; microcrystalline cellulose; gum, especially agar gum, and guar gum; alginic acid or salts thereof; acetates and citrates; sugars (especially sucrose, amylose, dextrose and lactose); aluminum oxide; synthetic polymers such as methacrylic acid-divinylbenzene copolymer, as well as effervescent disintegrating systems. Typical levels of disintegration aids in the inventive oral care preparations are from about 0.5% to about 15 % of the formulation, preferably from about 1% to about 5%.

The inventive oral care compositions may also contain one or more organoleptic enhancing agents. Organoleptic enhancing agents include humectants, sweeteners, surfactants, flavorants, colorants and effervescing agents.

Humectants serve to add body or “mouth texture” to a dentifrice. In addition to the previously mentioned sugar alcohols, suitable humectants include glycerin, polyethylene glycol (at a variety of different molecular weights), propylene glycol, and hydrogenated starch hydrolyzates, as well as mixtures of these compounds.

Sweeteners may be added to the dentifrice composition to impart a pleasing taste to the product. Suitable sweeteners include saccharin (as sodium, potassium or calcium saccharin), cyclamate (as a sodium, potassium or calcium salt), aspartame, acesulfane-K, thaumatin, neohisperidin dihydrochalcone, ammoniated glycyrrhizin, dextrose, maltodextrin, sucralose, fructose, levulose, sucrose, mannose, and glucose. Typical levels of sweeteners are from about 0% to about 5% of a dentifrice composition.

Surfactants are used in the compositions of the present invention to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Sodium lauryl sulfate is a preferred surfactant. The surfactant is typically present in the oral care compositions of the present invention in an amount of about 0.1 to about 15% by weight, preferably about 0.3% to about 5% by weight, such as from about 0.3% to about 2%, by weight.

Flavoring agents optionally can be added to dentifrice compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents consist chemically of mixtures of aldehydes, ketones, esters, phenols, acids, and aliphatic, aromatic and other alcohols.

Colorants may be added to improve the aesthetic appearance of the product. Suitable colorants are selected from colorants approved by appropriate regulatory bodies such as the FDA and those listed in the European Food and Pharmaceutical Directives and include pigments, such as TiO₂, and colors such as FD&C and D&C dyes.

The oral care product may also contain an effervescent agent to provide aesthetic properties to the tablet. Preferably effervescence is provided by reaction of a carbonate salt such as calcium carbonate, sodium carbonate, sodium bicarbonate, potassium carbonate or potassium bicarbonate with an acid such as citric acid, tartaric acid or malic acid.

In addition to calcium phosphate, the oral care tablet may contain additional abrasives. Suitable abrasives include precipitated and ground calcium carbonate, precipitated silica, such as Zeodent® silicas available from J. M. Huber Corporation, silica gel, calcium metasilicate, aluminum silicate, alumina, calcined alumina, bentonite, particulate thermosetting resins and other suitable abrasive materials known to a person of ordinary skill in the art. The abrasive may be used alone or in combination with other abrasives. Typical levels of abrasives in the inventive dentifrice formulation are from about 2% to about 60%, preferably from about 2% to about 10%.

Thickening agents are useful in the oral care compositions of the present invention to provide an aesthetically pleasing texture when the composition disintegrates in the mouth. Suitable thickening agents include silica thickeners such as J. M. Huber Corporation Zeodent® precipitated silica products and silica gels available from Davison Chemical Division of W. R. Grace Corporation, Baltimore, Md.; natural and synthetic clays such as hectorite clays, lithium magnesium silicate (laponite) and magnesium aluminum silicate (Veegum); starch; glycerite of starch, as well as mixtures of these compounds. Typical levels of thickening agents are from about 0% to about 15% of an oral care composition.

Therapeutic agents are optionally used in the compositions of the present invention to provide for the prevention and treatment of dental caries, periodontal disease and temperature sensitivity. Examples of therapeutic agents, without intending to be limiting, are fluoride sources, such as sodium fluoride, sodium monofluorophosphate, stannous fluoride, potassium fluoride, sodium fluorosilicate, ammonium fluorosilicate and the like; condensed phosphates such as tripolyphosphates, hexametaphosphates, trimetaphosphates and pyrophosphates; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quartemary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents may be used in dentifrice formulations singly or in combination at a therapeutically safe and effective level.

Preservatives may be also be optionally added to the compositions of the present invention to prevent bacterial growth. Suitable preservatives approved for use in oral compositions such as methylparaben, propylparaben and sodium benzoate may be added in safe and effective amounts.

The oral care products may additionally contain other optional ingredients typically used in tablet making such as glidants to provide even flow to the granulation to be tabletted, e.g. amorphous silica such as Zeopharm® 80 (J. M. Huber Corporation, Edison, N.J.) and Cab-O-Sil® M5 (Cabot Corporation, Billerica, Mass.); die release aids, also known as lubricants, such as magnesium stearate (available as HYQUAL® NF from Mallinckrodt, Inc., St. Louis, Mo.) to enable tablets to be released from within the tablet machine die, anti-adherants, such as stearic acid, to facilitate separation of tablets from punch faces; and fillers such as microcrystalline cellulose, such as Avicel 101 (FMC Biopolymers, Philadelphia, Pa.) and Omnicel 102 (Functional Foods, Englishtown, N.J.).

All tablet formulation ingredients, except the lubricant, are weighed together and mixed. Thereafter, the lubricant is geometrically diluted with the just prepared tablet mixture and then added back to the mixture. This step is typically necessary to homogeneously incorporate the hydrophobic lubricant into the tablet mixture.

The tablets are then manufactured by using a tableting compacting process. A standard single stroke or a rotary press may be used. The tablets prepared according to this invention may be of any geometrical shape, such as round, square, triangular or caplet-shaped, and of any size suitable for human or animal use.

The invention will now be described in more detail with respect to the following, specific, non-limiting examples.

### Oral Care Tablet Preparation

Oral care tablets were prepared by weighing all formulation ingredients together, except the lubricant magnesium stearate, on a weighing pan. Typically, a tablet formulation was 300 g to 500 g total weight, in order to prepare multiple tablets for testing. The combined ingredients were passed through a 20 mesh (850 μm) sieve to remove any lumps and then bag blended, by gentle inversion in a plastic bag for about 30 seconds of the formulation ingredients previously weighed. The resulting mixture was transferred to a PK-V blender (twin shell dry blender model 014-215-0053, available from Patterson Kelly, East Stroudsburg, Pa.) and mixed for 10 minutes. The magnesium stearate lubricant was then geometrically diluted with the mixture and then added back to the PK blender and all ingredients mixed together for an additional 5 minutes.

Tablets were formed from the resulting formulation on a 8-station Piccola rotary tablet press available from Riva S.A., Argentina, fitted with 10 mm standard concave die punches compacting over a range of compression forces. Tablet weight was set at 400 mg by adjusting the tablet press.

### Tablet Test Methods

All tablets were prepared 24 hours before testing hardness, disintegration time and friability.

Tablet hardness (H) expressed in kP, for each formulation, was measured on 5 tablets utilizing a Erweka TBH30 instrument (Milford, Conn.) and the result reported was an average of 5 measurements.

Tablet disintegration time was determined according to the USP test for uncoated tablets by placing 6 tablets (with each tablet in a separate tube) in an Erweka ZT72 disintegrator (Milford, Conn.). The tablets were repeatedly immersed in 37° C. deionized water at a rate of 30 strokes/min until the tablets disintegrated, as detected and recorded by the instrument. The reported result was an average of the 6 measurements.

Tablet friability was determined by placing 10 tablets in a Distek, Inc. Friabilator DF-3 (North Brunswick, N.J.) set for 100 revolutions. The % friability is calculated from the amount of tablet weight lost (friable) by weighing the tablets before and after rotation.

### EXAMPLE 1

In this example, basic oral care tablet formulations were made with the abrasives dicalcium phosphate dihydrate (DCPD) and tricalcium phosphate (TCP) a super disintegrant and a sugar alcohol. These formulations were prepared according to the procedure described above with the amounts of ingredients identified in Table 1.

TABLE 1
Tablet Composition

| | | Formulation No. | | | | |
|---|---|---|---|---|---|---|
| Ingredient | Source | 1 | 2 | 3 | 4 | 5 |
| DCPD, % | Penwest, Patterson, NY | 27 | 27 | 0 | 27 | 0 |
| Emcompress | | | | | | |
| TCP, % | Chemische Fabrik | 0 | 0 | 27 | 0 | 27 |
| Tri-Cafos P | Budenheim Germany | | | | | |
| Mannitol, % | Roquette Freres, | 69.25 | 49.25 | 49.25 | 35 | 20 |
| Pearlitol 200SD | Lestrem, France | | | | | |
| compressible sugar, % | Chr. Hansen, | 0 | 20 | 20 | 34.25 | 49.25 |
| Nu-Tab 4000 | Vineland, NJ | | | | | |
| Crospovidone, % | ISP Technologies, | 3 | 3 | 3 | 3 | 3 |
| Polyplasdone ® XL | Inc., Wayne, NJ | | | | | |
| Magnesium Stearate, % | Mallinckrodt, Inc., | 0.75 | 0.75 | 0.75 | 0.75 | 0.75 |
| Hyqual NF | St. Louis, MO | | | | | |

Tablets weighing 400 mg each were prepared according to the procedure described above. Each formulation was compressed into tablets at three different compression forces. This set of experiments compared the performance of the inventive oral care tablets formulated with a calcium phosphate, a super disintegrant, and varying amounts of a sugar alcohol. The tablet hardness (H), disintegration time (DT) and Friability were determined according to the procedures described above for tablets pressed at different compression forces with the results summarized in Table 2 below.

TABLE 2
Tablet Properties

| Formulation | Compression Force | Hardness | DT | Friability |
|---|---|---|---|---|
| No. | (kN) | (kP) | (sec) | % |
| 1 | 3.7 | 2.55 | 11 | — |
| 1 | 7.7 | 6.8 | 11 | — |
| 1 | 10 | 9.85 | 12 | — |
| 2 | 5.5 | 3.30 | 7 | 1.01 |
| 2 | 10.0 | 7.89 | 12 | 0.41 |
| 2 | 13.8 | 11.56 | 25 | 0.20 |
| 3 | 3.8 | 2.77 | 12 | 1.45 |
| 3 | 7.5 | 6.67 | 18 | 0.37 |
| 3 | 10.2 | 9.62 | 33 | 0.25 |
| 4 | 5.3 | 2.98 | 9 | 1.19 |
| 4 | 10.0 | 7.42 | 20 | 0.33 |
| 4 | 12.9 | 9.83 | 40 | 0.23 |
| 5 | 4.0 | 2.85 | 14 | 1.05 |
| 5 | 7.2 | 7.16 | 23 | 0.43 |
| 5 | 9.2 | 9.40 | 37 | 0.40 |

It is seen from the data above that the inventive tablets when compressed to a tablet hardness of about 7 kP had a friability of less than 1% and disintegrated in less than 40 seconds. This small friability percentage reflects the fact that the tablets are strong and have excellent physical integrity. This means that they can remain intact during the periods of storage and transportation until being finally delivered to the consumer. (The above data in Table 2 is discussed further, below.)

### COMPARATIVE EXAMPLE 1

Tablets were prepared according to the procedure described above from the formulations given in Table 3 below for comparative purposes. Formulation A did not contain a sugar alcohol and the Formulation B did not contain a super disintegrant.

TABLE 3
Tablet Formulations

| | Formulation | Formulation |
|---|---|---|
| | A | B |
| DCPD, % | 27 | 27 |
| Emcompress ® | | |
| Mannitol, % | 0 | 72.25 |
| Pearlitol 200SD | | |
| Compressible sugar NF, % | 69.25 | 0 |
| Nu-Tab 4000 | | |
| Crospovidone, % | 3 | 0 |
| Polyplasdone ® XL | | |
| Magnesium Stearate, % | 0.75 | 0.75 |
| Hyqual NF | | |

The tablets were prepared by compression at 3 different compression forces and tested for hardness and disintegration time according to the methods described above with the results are summarized in Table 4 below.

TABLE 4
Tablet Properties

| Formulation | Compression Force | Hardness | DT |
|---|---|---|---|
| No. | (kN) | (kP) | (sec) |
| A | 3 | 2.38 | 90 |
| A | 5.2 | 4.83 | 97 |
| A | 6.6 | 7.58 | 169 |
| B | 4.10 | 4.24 | 94 |
| B | 8.40 | 9.50 | 278 |
| B | 11.40 | 12.54 | 281 |

It is seen that the comparative tablets without mannitol (Formulation A) and the comparative tablets without a super disintegrant (Formulation B) took more than 90 seconds to disintegrate. By contrast, all of the tablets prepared according to the present invention (see Table 2, above) disintegrated in less than 40 seconds. Thus, the tablets prepared according to the present invention disintegrate much faster than the comparative, prior art tablets.

Moreover, this improved disintegration performance is obtained without compromising the physical integrity of the tablet. As can be seen in Tables 2 and 4, the hardness of the tablets prepared according to the present invention is comparable to the hardness of the comparative prior art tablets. This indicates that the tablets will be more durable during manufacture, storage and transport and have a greater chance of finally reaching the consumer intact.

### EXAMPLE 2

In this example, oral care tablet formulations were made with the abrasives dicalcium phosphate dihydrate (DCPD) or tricalcium phosphate (TCP), the sugar alcohols mannitol and sorbitol, a super disintegrant blend of crospovidone and Explotab and other ingredients typically found in oral care products. These formulations were prepared according to the procedure described above from the amounts of ingredients given in Table 5 below.

TABLE 5
Tablet Formulations

| | Formulation 6 | Formulation 7 |
|---|---|---|
| DCPD, % | 38.00 | 0 |
| TCP, % | 0 | 38.00 |
| Mannitol, % | 25.74 | 25.74 |
| Sorbitol, % | 10.00 | 10.00 |
| Polyplasdone ® XL/ | 5.00 | 5.00 |
| Explotab ® (1:1 Blend), % | | |
| Sodium lauryl sulfate, % | 1.00 | 1.00 |
| Avicel ® 101 MCC, % | 13.50 | 13.50 |
| Sodium fluoride, % | 0.01 | 0.01 |
| Cab-O-Sil M-5, % | 1.00 | 1.00 |
| Sucralose, % | 1.50 | 1.50 |
| Flavor, % | 3.50 | 3.50 |
| Magnesium stearate, % | 0.75 | 0.75 |

The DCPD used was Emcompress available from Penwest, Patterson, N.Y.; the TCP was Tri-Cafos P available from Budenheim, Germany; the mannitol was Pearlitol 200SD available from Roquette Freres, Lestem, France; the super disintegrant was a 1:1 blend of Polyplasdone® XL (crospovidone, available from ISP Technologies, Inc., Wayne, N.J.) and Explotab® (sodium starch glycolate available from Penwest, Patterson, N.J.); Avicel 101 microcrystalline cellulose (MCC) available from FMC Biopolymers, Philadelphia, Pa.; and Cab-O-Sil® M5 silica glidant available from Cabot Corporation, Billerica, Mass.

Tablets were prepared from Formulations 6 and 7 according to the procedure described above, compressed at three different compression forces and tablet properties of hardness, disintegration time (DT) and friability determined according to the methods described above with the results summarized in Table 6 below.

TABLE 6
Tablet Properties

| Formulation | Compression | Hardness | DT | |
|---|---|---|---|---|
| No. | Force (kN) | (kP) | (s) | % Friability |
| 6 | 4.70 | 2.83 | 13 | 0.952 |
| 6 | 9.00 | 7.54 | 44 | 0.267 |
| 6 | 12.20 | 10.36 | 86 | 0.194 |
| 7 | 3.50 | 2.24 | 26 | 1.595 |
| 7 | 7.20 | 6.73 | 18 | 0.484 |
| 7 | 9.20 | 8.95 | 15 | 0.357 |

It is seen in Table 6, the tablets containing DCPD (Formulation 6) showed excellent disintegration time while at the same time having an excellent physical integrity/intactness as indicated by their friability of less than 1%. It is true that for Formulation 6 the disintegration time increased with increasing hardness, however, disintegration times were still relatively brisk: the longest being 86 seconds.

The tablets made with TCP abrasive (Formulation 7) showed reduced disintegration time as tablet hardness increased, such fast disintegration of tablets made from TCP would have been unexpected to a person of ordinary skill in the art. The tablets with the faster disintegration times also had extremely low % friability.

### EXAMPLE 3

In this example, oral care effervescent tablets were made with the abrasive phosphate dihydrate (DCPD) or tricalcium phosphate (TCP); the sugar alcohol super disintegrant of either crospovidone or a blend of crospovidone and and sodium bicarbonate and citric acid, which provide an effervescent effect acted with water or saliva. Additionally, these tablets contained other ingredients normally found in oral care dentifrices. These tablets were prepared according to the procedure described above with the amounts of ingredients identified in Table 7.

TABLE 7
Tablet Formulations

| | Formu- | Formu- | Formu- | Formu- |
|---|---|---|---|---|
| | lation 8 | lation 9 | lation 10 | lation 11 |
| DCPD, % | 20 | 16 | 0 | 0 |
| Emcompress | | | | |
| TCP, % | 0 | 0 | 20 | 16 |
| Tri-Cafos P | | | | |
| Sorbitol, % | 8 | 8 | 8 | 8 |
| Polyplasdone ® | 14 | 0 | 14 | 0 |
| XL, % | | | | |
| Polyplasdone ® | 0 | 11 | 0 | 11 |
| XL/Explotab | | | | |
| (1:1 Blend), % | | | | |
| Avicel 101 MCC, % | 16.4 | 20 | 16.4 | 20 |
| Sodium | 20 | 20 | 20 | 20 |
| bicarbonate, % | | | | |
| Citric Acid, % | 10 | 10 | 10 | 10 |
| Zeodent 9175 | 3 | 3 | 3 | 3 |
| silica abrasive, % | | | | |
| Cab-O-Sil M-5, % | 1 | 1 | 1 | 1 |
| Aspartame, % | 3 | 3 | 3 | 3 |
| Flavor, % | 3 | 3 | 3 | 3 |
| Sodium lauryl | 1 | 1 | 1 | 1 |
| sulfate, % | | | | |
| Papain, % | 0.1 | 0 | 0.1 | 0 |
| Sodium fluoride, % | 0.01 | 0 | 0.01 | 0 |
| Cetyl pyrridinium | 0 | 0.5 | 0 | 0.5 |
| chloride, % | | | | |
| Sodium tri- | 0 | 3 | 0 | 3 |
| polyphosphate, % | | | | |
| Magnesium | 0.5 | 0.5 | 0.5 | 0.5 |
| Stearate, % | | | | |

Formulations 8 and 9 contained DCPD abrasive and Formulations 10 and 11 contained TCP abrasive. Formulations 8 and 10 contained all the same amounts of other ingredients as do Formulations 9 and 11. The difference in these 2 sets of formulations (8 and 10 verses 9 and 11) is the type and amount of super disintegrant. Formulations 8 and 10 contain the super disintegrant crospovidone while Formulations 9 and 11 contain a super disintegrant mixture of crospovidone and sodium starch glycolate. Tablets weighing 400 mg each were prepared from these formulations according to the procedure described above and several tablet properties were determined according to the methods described above.

TABLE 8
Tablet Properties

| | Formulation | Formulation | Formulation | Formulation |
|---|---|---|---|---|
| | 8 | 9 | 10 | 11 |
| Compression | 9.2 | 10.3 | 7.9 | 9.2 |
| Force, kN | | | | |
| Hardness, kP | 2.21 | 2.75 | 2.55 | 2.62 |
| DT, (seconds) | 49 | 67 | 48 | 53 |
| % Friability | 0.907 | 0.936 | 0.568 | 0.902 |

It is seen in Table 8 that the tablets had fast disintegration times, while at the same time having very low friability, in every case of less than 1%.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A rapidly disintegrating oral care tablet comprising:
a calcium phosphate;
a super disintegrant; and
a sugar alcohol;
wherein the tablet has a friability of less than about 2% and disintegrates when immersed in water in less than about 60 seconds.

2. The oral care tablet according to claim 1, wherein the calcium phosphate is selected from one or more of dicalcium phosphate, dicalcium phosphate dihydrate, tricalcium phosphate, calcium pyrophosphate, and calcium polymetaphosphate.

3. The oral care tablet according to claim 1, wherein the tablet comprises about 10% to about 80 wt % of calcium phosphate.

4. The oral care tablet according to claim 1, wherein the super disintegrant is selected from one or more of sodium starch glycolate, croscarmellose sodium, and crospovidone.

5. The oral care tablet according to claim 1, wherein the tablet comprises about 1 wt % to about 30 wt % of the super disintegrant.

6. The oral care tablet according to claim 1, wherein the tablet comprises about 1 wt % to about 3 wt % of the super disintegrant

7. The oral care tablet according to claim 1, wherein the sugar alcohol is selected from one or more of sorbitol, mannitol, xylitol, erythritol, maltitol, and lactitol.

8. The oral care tablet according to claim 1, wherein the tablet comprises about 20 wt % to about 80 wt % of the sugar alcohol.

9. The oral care tablet according to claim 1, wherein the tablet friability is less than 1%.

10. The oral care tablet according to claim 1, wherein the tablet, when added to water at 37° C. disintegrants in less 40 seconds.

11. The oral care composition according to claim 1, further comprises one or more ingredients selected from the group consisting of: organoleptic enhancing agents, abrasives, disintegration aids, preservatives, therapeutic agents and thickening agents.

12. The oral care composition according to claim 11, wherein the organoleptic enhancing agent comprises one or more ingredients selected from the group consisting of humectants, sweeteners, flavorants, surfactants, colorants and effervescent agents.

13. A rapidly disintegrating oral care tablet comprising:
about 10 wt % to about 80 wt % calcium phosphate;
about 1 wt % to about 15 wt % super disintegrant;
about 20 wt % to about 80 wt % sugar alcohol; and
about 0.1 wt % to about 5 wt % surfactant;
wherein the tablet has a friability of less than about 2% and disintegrates when immersed in water in less than about 60 seconds.

14. A rapidly disintegrating oral care tablet according to claim 13, fuirther comprising a flavorant.
